SEQUENCE LISTING (1 sequence)

SEQ ID NO: 1 - DNA, 26 bp, artificial sequence
  hairpin
aaaaccaaaaatttttttttggtttt

(19) United States
(12) Patent Application Publication
(10) Pub. No.: US 2018/0355441 A1
(43) Pub. Date: Dec. 13, 2018

(54) VISUAL DETECTION OF PLATINATED DNA LESIONS FROM A CLICKABLE CISPLATIN PROBE USED AS DIAGNOSTIC TOOL OR TO IDENTIFY SYNERGISTIC TREATMENTS

(71) Applicants: CENTRE NATIONAL DE LA RECHERCHE SCIENTIFIQUE, PARIS (FR); INSTITUT CURIE, PARIS CEDEX 05 (FR); UNIVERSITY OF TEXAS AT AUSTIN, AUSTIN, TX (US); INTITUTE NATIONAL DE LA SANTE ET DE LA RECHERCHE MEDICALE, PARIS (FR)

(72) Inventors: RAPHAEL RODRIGUEZ, VERS-PONT-DU-GARD (FR); EMMANOUIL ZACHARIOUDAKIS, ORSAY (FR); LAVANIYA KUNALINGAM, SARCELLES (FR); ALEXANDRA BARTOLI, MARSEILLE (FR); KYLE MILLER, AUSTIN, TX (US); POONAM AGARWAL, AUSTIN, TX (US)

(21) Appl. No.: 16/061,665

(22) PCT Filed: Dec. 15, 2016
(86) PCT No.: PCT/EP2016/081166

§ 371 Date: Jun. 13, 2018

(30) Foreign Application Priority Data
Dec. 15, 2015 (EP) 15307009.9

(51) Int. Cl. C12Q 1/6886; C07F 15/00

(52) U.S. Cl. CPC C12Q 1/6886; C07F 15/0093; C12Q 2600/106; C12Q 2523/101; C12Q 2600/118; C12Q 2600/136; C12Q 2600/142; C12Q 2600/112

(57) ABSTRACT

The present invention relates to a new compound for visualizing DNA-platinum crosslink, and its use as a research tool and in screening method for identifying candidate drug to be used in combination with platinating compounds such as cisplatin, carboplatin, and oxaliplatin. The project leading to this application has received funding from the European Research Council (ERC) under the European Union's Horizon 2020 research and innovation program (grant agreement No [647973]).

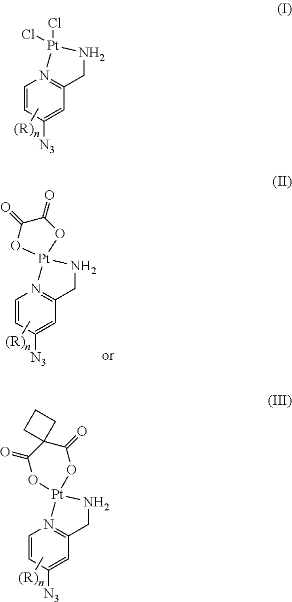

### FIELD OF THE INVENTION

The present invention relates to the field of oncology and laboratory tools. It relates to new compounds suitable for visualizing platinated DNA crosslinks.

### BACKGROUND OF THE INVENTION

Cisplatin is one of the most effective broad-spectrum anticancer drugs. Platinating compounds such as cisplatin, carboplatin, and oxaliplatin are still front-line clinical therapies and constitute part of the treatment regimen for patients with many types of cancers, including head and neck, testicular, ovarian, cervical, lung, colorectal and relapsed lymphoma.

Platinum drugs form stable covalent bonds with guanine residues to produce DNA crosslinks. These lesions interfere with replication and transcription, thereby leading to DNA breaks and cell death. The cellular response to cisplatin is pleiotropic and inherently complex. For example, platinated DNA lesions (DNA-Pt) can be processed by diverse repair mechanisms including nucleotide excision repair (NER), base excision repair (BER) and DNA crosslink repair involving the Fanconi anemia pathway, all of which may be influenced by DNA sequences and chromatin features. Alternatively, intra-strand lesions can be bypassed by low-fidelity DNA polymerases through a mechanism known as translesion synthesis (TLS), enabling continued replication in the presence of platinated DNA lesions.

Patients usually have a good initial response to cisplatin-based chemotherapy but later relapse, because the development of cisplatin resistance, either acquired or intrinsic, markedly reduces its clinical effectiveness.

Ding et al (2013, Angew Chem Int Ed Engl, 52, 3350-54) developed a method to probe DNA targeted platinum by using post-labeling of platinum-acridine hybrid by click reactions with an alkyne-fluorophore with cell-free DNA and in whole cancer cells. However, the platinum-acridine hybrid is structurally different from cisplatin. It is noteworthy that the presence of the double strand DNA intercalator (i.e. acridine) likely dominates genome targeting by this dimer to induce a distinct genomic response compared to cisplatin. Displacement of the azide-containing acridine upon crosslink formation with DNA is also expected to lead to a chemical labeling reflecting the cellular localization of the acridine itself as opposed to DNA-Pt. Furthermore, it does not present the same cytotoxicity than cisplatin (up to 500-times more cytotoxic). Therefore, the platinum-acridine hybrid, as shown below, does not recapitulate the clinically relevant drug cisplatin.

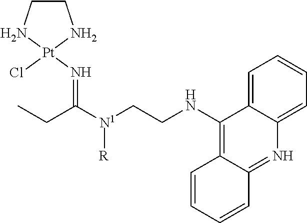

with R being (CH₂)₂CONH(CH₂)₂N₃

White et al (2013, J Am Chem Soc, 135, 11680-11683) discloses picazoplatin, an azide-containing platinum (II) derivative. The authors developed this derivative for identifying, visualizing in vitro of a gelcellular targets of platinum complexes. They added an azide moiety to picoplatin drug. Picazoplatin is labeled by click reactions with an alkyne-fluorophore.

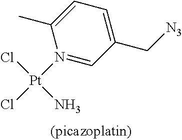

Wirth et al (2015, J Am Chem Soc, 137, 15169-75) and Moghaddam et al (2015, Dalton Trans, 44, 3536-3538) disclose other derivatives of cisplatin which are either azide-appended or alkyne-appended. In this article, the authors compared the following two compounds

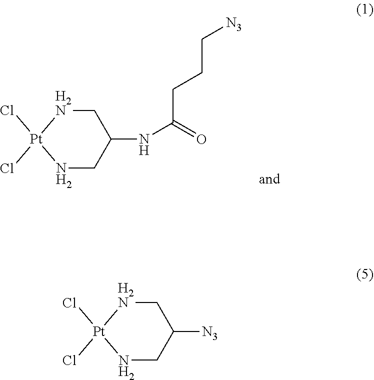

They concluded that compound (1) presents a higher reactivity than compound (5) because of an increased steric accessibility and potentially of the presence of the amide bound.

However, despite these new developments, there is still a strong need of molecule suitable for recapitulating the DNA-platinum crosslinks occurring with the platinum drugs. Indeed, visual detection (and pull-down) of DNA-Pt crosslinks with high resolution at the single-cell level could provide the means to monitor proteins at sites of lesions and to identify molecules with a propensity to modulate targeting with cisplatin in an unbiased manner. In addition, a significant challenge consists of functionalizing the inorganic platinum substrate with an organic moiety without altering the reactivity of the metal towards DNA, and optionally maintaining acceptable biological activity.

Therefore, any new method or tool useful for predicting or studying cisplatin resistance or for identifying a molecule capable of overcoming the cisplatin resistance would be of interest in this regard.

### SUMMARY OF THE INVENTION

The inventors developed a new compound, which is an analog of platinum drugs, mimicking the effect of platinum drugs and creating detectable DNA-platinum crosslinks, thereby enabling detection of platinated DNA lesions in cells. This compound can be used in a method for screening or identifying molecules to be used in combination with platinum drugs in order to prevent or delay the occurrence of resistance to platinum drugs or to overcome or reduce resistance to platinum drugs.

Accordingly, the present invention relates to a compound of formula (I), (II) or (III)

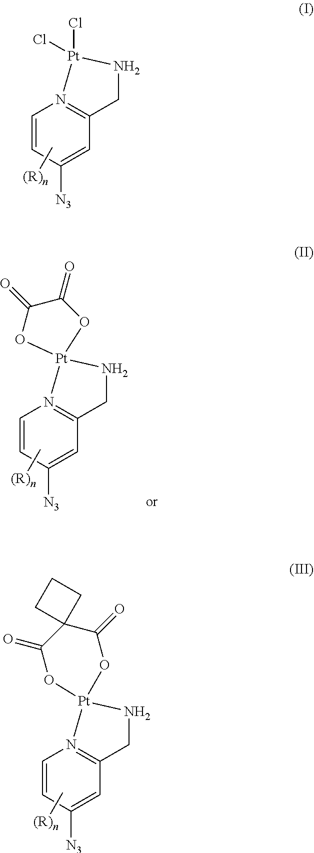

wherein n is an integer from 0 to 3 and R, independently, is selected from the group consisting of a group hydroxyl, cyano, amino, carboxyl, guanidinyl, —COOR′, —NHR′, —NR′R″, —N⁺R′R″R′″, —COR′, —CONHR′, —NHCOR′, phosphate, C(1-6) alkyl, C(2-6) alkenyl, C(1-6) alkoxy, said(1-6) alkyl, C(2-6) alkenyl, and C(1-6) alkoxy being optionally substituted by one or several groups selected from hydroxyl, cyano, amino, carboxyl, guanidinyl, —COOR′, —NHR′, —NR′R″, —N⁺R′R″R′″, —COR′, —CONHR′, —NHCOR′, aryl optionally substituted by methoxy or hydroxy, R′, R″ and R′″ being independently H or a C(1-6) alkyl.

Preferably, n is 1 and R is in position meta in respect to N₃.

Preferably, R is a charged radical at neutral pH, preferably a positively charged radical.

More preferably, R is a C(1-6) alkyl substituted by a group selected from hydroxyl, carboxyl, amino, guanidinyl, —NHR′, —NR′R″, —N⁺R′R″R′″, —CONHR′ or an aryl, optionally substituted by a hydroxyl or a methoxy.

Alternatively, n is 0 and the formula is (I).

In another alternative, n is 0 and the formula is (II).

The present invention also relates to a kit comprising a compound according to the present invention and a label bearing an alkyne group, preferably a fluorescent label or a biotinylated label.

The present invention further relates to the in vitro use of a compound according to the present invention or of a kit as disclosed herein as a research tool, in particular for visualizing platinated DNA crosslinks in cells or for recovering platinum-bound DNA. In addition, it relates to the in vitro use of a compound according to the present invention or of a kit as disclosed herein for identifying or screening a molecule capable of preventing or delaying the occurrence of resistance to platinum drugs or to overcome or reduce resistance to platinum drugs or for predicting a sensitivity or resistance to a platinum drug in a patient.

The present invention relates to an in vitro method for visualizing platinated DNA crosslinks in cells, the method comprising:
contacting a cell with a compound according to the present invention;
contacting said cell with a label bearing an alkyne group, preferably a fluorescent label, optionally in presence of copper; and
detecting the label in said cell, preferably the fluorescent label.

Preferably, before the step of contacting said cell with a label bearing an alkyne group, the cell is permeabilized and then fixed.

The present invention relates to an in vitro method for predicting a resistance or sensitivity of a tumor in a patient to a platinum drug, comprising
carrying out the method for visualizing platinated DNA crosslinks in cells as disclosed herein with a cell from a tumor sample from the patient;
measuring the labeling and optionally comparing the labeling to a reference level; and
determining the resistance or sensitivity to a platinum drug of the tumor in the patient based on the intensity of the labeling, the sensitivity being proportional to the intensity of the labeling.

The present invention relates to an in vitro method for identifying or screening a molecule capable of preventing or delaying the occurrence of resistance to platinum drugs or to overcome or reduce resistance to platinum drugs, the method comprising:
contacting a cell with a compound according to the present invention and with a candidate molecule, wherein the contact with the compound can be after, simultaneously, or before the contact with the candidate molecule;
contacting said cell with a label bearing an alkyne group, preferably a fluorescent label, optionally in presence of copper;
measuring the labeling;
optionally comparing the intensity of the labeling in the presence and the absence of the candidate molecule;
selecting the candidate molecule if the intensity of the labeling is increased and/or the morphology of foci is different in the presence of the candidate molecule when compared to the intensity of the labeling in absence of candidate molecule.

### DETAILED DESCRIPTION OF THE INVENTION

In the present invention, the inventors report an original strategy to chemically label an analog of platinum drugs in cells. More particularly, the analog of platinum drugs is able to form DNA-platinated crosslinks in cells in a similar manner than platinum drugs and can be easily labeled in situ. The present technology was successfully implemented to visualize platinated DNA crosslinks in cells. It was further employed in cancer cells to screen for small molecules that could affect genome targeting with platinum drugs, in particular cisplatin. By implementing this strategy, the inventors have identified the clinically approved drug vorinostat, a known inhibitor of histone deacetylases, as a small molecule that induced hyper loading of platinum onto specific genomic loci; discovered that these clusters of lesions co-localized with translesion synthesis factors and activated this pathway and found that translesion synthesis no longer acted as a bypass/resistance mechanism but instead promoted apoptosis after co-treatment with cisplatin and HDACi (histone deacetylase inhibitor). This study has led to a new model whereby inhibition of histone deacetylases increases local platinum loading, where lesions act as roadblocks to translesion synthesis, which triggers apoptosis.

Therefore, the present invention provides a new compound useful as a research tool for studying and understanding cellular responses to platinum drugs. This compound is also useful as a screening tool for identifying molecules to be used in combination with platinum drugs in order to prevent or delay the occurrence of resistance to platinum drugs or to overcome or reduce resistance to platinum drugs.

Platinum Drug Analog

The present invention relates to a compound useful as a platinum drug analog.

By “platinum drug” is intended a class of platinum-based antineoplastic drugs which are chemotherapeutic agents used for treating cancer. They are coordination complexes of platinum. Non exhaustively, the class of drugs includes cisplatin, cisplatinum, carboplatin, oxaliplatin, satraplatin, picoplatin, nedaplatin, and triplatin.

The compound has one of the following formulae (I), (II) or (III):

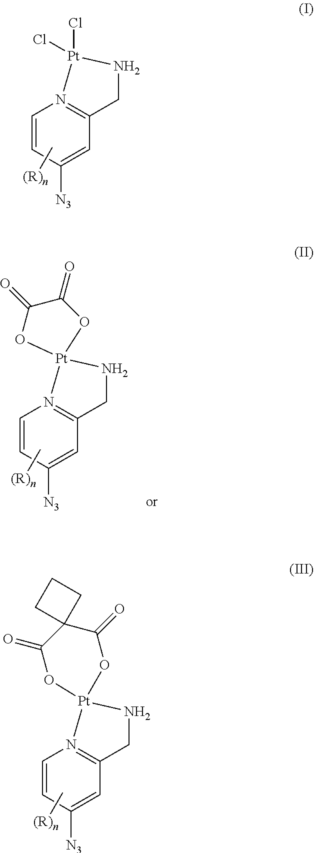

wherein n is an integer from 0 to 3 and R, independently, is selected from the group consisting of a group hydroxyl, cyano, amino, carboxyl, guanidinyl, —COOR′, —NHR′, —NR′R″, —N′R′R″R′″, —COR′, —CONHR′, —NHCOR′, phosphate, C(1-6) alkyl, C(2-6) alkenyl, C(1-6) alkoxy, said(1-6) alkyl, C(2-6) alkenyl, and C(1-6) alkoxy being optionally substituted by one or several groups selected from hydroxyl, cyano, amino, carboxyl, guanidinyl, —COOR′, —NHR′, —NR′R″, —N⁺R′R″R′″, —COR′, —CONHR′, —NHCOR′, phosphate, aryl optionally substituted by methoxy or hydroxyl, R′, R″ and R′″ being independently H or a C(1-6) alkyl.

Compounds of formula (I) are derived from cisplatin whereas the compounds of formulae (II) and (III) are derived from oxaliplatin and carboplatin, respectively.

n can be 0, 1, 2 or 3. Preferably, n is 0 or 1. In one preferred embodiment, n is 0. In another preferred embodiment, n is 1.

In a specific embodiment, the compound has the structure of formula (I) wherein n is 0. This compound is called azidocycloplatin (ACP) or 2-aminomethylpyridine(dichloro)platinum(II) azide (APPA).

In another specific embodiment, the compound has the structure of formula (II) wherein n is 0. This compound is called 2-aminomethylpyridine (oxalo) platinum (II) azide (APPOA).

When n is 1, R can be in position ortho or meta with respect to the azide, N₃. Preferably, R is in position meta with respect to the azide, N₃. In this embodiment, the compound has one of the following formulae (Ia), (IIa) or (IIIa):

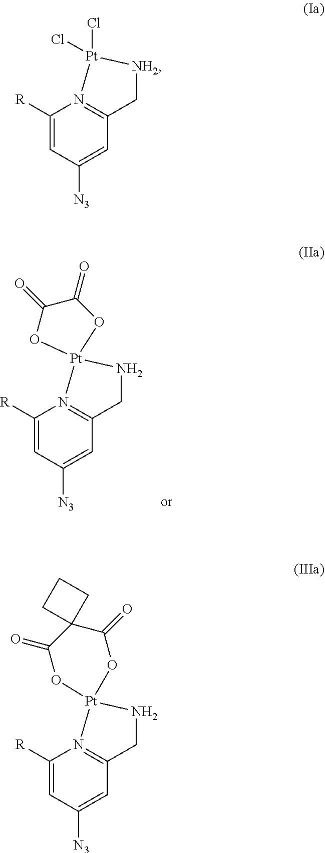

Respectively, the compounds of formula (I) or (la) are analogs of cisplatin, the compounds of formula (II) or (IIa) are analogs of oxaliplatin, and the compounds of formula (III) or (IIIa) are analogs of carboplatin.

In a particular embodiment, R is selected so as to improve the solubility of the compound in comparison of the compound devoid of R radical. Accordingly, R can be a charged radical at neutral pH, negatively or positively charged. More preferably, R is a positively charged radical, especially at a neutral pH. Indeed, the positive charge could be an advantage when considering the negative charge of DNA.

Preferably, R′, R″ and R′″ are independently H or a C(1-3) alkyl, more preferably are H, methyl or ethyl, still more preferably are H or methyl.

In a particular embodiment, R is a C(1-6) alkyl substituted by a group selected from hydroxyl, carboxyl, amino, guanidinyl, —NHR′, —NR′R″, —N⁺R′R″R′″, —CONHR′ or an aryl, optionally substituted by a hydroxyl or a methoxy.

In a specific embodiment, R is —(CH2)p-A, with A being selected from the group consisting of —OH, —COOH, —NH2, —NHMe, —N(Me)₂, —N⁺(Me)₃, —CONH, —NHCOMe, guanidinyl and a phenyl optionally substituted by a hydroxyl, and with p being 1, 2, 3 or 4. Preferably, p is 2, 3 or 4.

According to the present invention, the terms below have the following meanings:

The terms mentioned herein with prefixes such as for example C₁-C₃ or C₁-C₆ can also be used with lower numbers of carbon atoms such as C₁-C₂ or C₁-C₅. If, for example, the term C₁-C₃ is used, it means that the corresponding hydrocarbon chain may comprise from 1 to 3 carbon atoms, especially 1, 2 or 3 carbon atoms. If, for example, the term C₁-C₆ is used, it means that the corresponding hydrocarbon chain may comprise from 1 to 6 carbon atoms, especially 1, 2, 3, 4, 5 or 6 carbon atoms.

The term “alkyl” refers to a saturated, linear or branched aliphatic group. The term “(C₁-C₃)alkyl” more specifically means methyl (also called “Me”), ethyl (also called “Et”), propyl, or isopropyl, the term “(C₁-C₆)alkyl” more specifically means methyl, ethyl, propyl, isopropyl, butyl, isobutyl, tert-butyl or propyl, pentyl or hexyl.

The term “alkoxy” or “alkyloxy” corresponds to the alkyl group defined hereinabove bonded to the molecule by an —O— (ether) bond. (C₁-C₃)alkoxy includes methoxy, ethoxy, propyloxy, and isopropyloxy. (C₁-C₆)alkoxy includes methoxy, ethoxy, propyloxy, isopropyloxy, butyloxy, isobutyloxy, tert-butyloxy, pentyloxy and hexyloxy. The term (C₁-C₆)ₙpolyalkyloxy corresponds to n (C₁-C₆)alkyloxy bounded thereby forming a linear poly(C₁-C₆)alkylene glycol chain, preferably a linear polyethylene glycol chain. Preferably, n is 1<n<6.

The term “aryl” is mono- or bi-cyclic aromatic hydrocarbons having from 6 to 12 carbon atoms, optionally substituted. Aryl may be a phenyl (also called “Ph”), biphenyl or naphthyl. In a preferred embodiment, the aryl is a phenyl.

—COOR′ refers to a carboxyl group. —NHR′, —NR′R″, —N⁺R′R″R′″ respectively refer to secondary, tertiary and quaternary amine. —COR′ refers to an acyl. —CONHR′ and —NHCOR′ refer to amide.

The compounds of the present invention can be synthesized by methods known by the person skilled in the art, and in particular by using the synthesis schema detailed below.

The present invention relates to a composition and kit comprising a compound of the present invention.

### Methods and Kits of the Present Invention

The compound of the invention is suitable for forming DNA-platinum detectable crosslinks, then for labeling DNA-platinum crosslinks or DNA sites susceptible to be platinated. Therefore the present invention relates to the use of any compound of the present invention as detailed above or kit comprising it as a research tool, especially for labeling DNA-platinum crosslinks or localizing the genomic sites comprising DNA-platinum crosslinks, and in particular for visualizing platinated DNA crosslinks in cells or for recovering platinum-bound DNA, in particular for sequence analysis. It also relates to the use of any compound of the present invention as detailed above or kit comprising it for identifying or screening a molecule capable of preventing or delaying the occurrence of resistance to platinum drugs or to overcome or reduce resistance to platinum drugs.

Indeed, by carrying the click chemistry, especially the copper-catalyzed azide-alkyne cycloaddition (CuAAC) or the strain-promoted alkyne-azide cycloaddition (SPAAC), a label can be covalently linked to the azide (N₃) group of the compound of the present invention. This chemistry, also referred as “bioorthogonal” or “biocompatible”, is compatible with the presence of a plurality of biological entities and can be carried out in cells.

The copper-catalyzed azide-alkyne cycloaddition (CuAAC) necessitates the presence of copper(I) catalyzer. It can be provided by the use of copper(II) precursors with a reducing agent (sodium ascorbate or p-hydrochinone for instance), by copper(I) salts or by pre-formed copper(I) complexes.

In an alternative specific aspect, biocompatible or biorthogonal click reactions encompass metal-free click-reactions (i.e. which do not require metal catalysts). An example of metal-free click reactions with cycloalkyne is depicted hereunder:

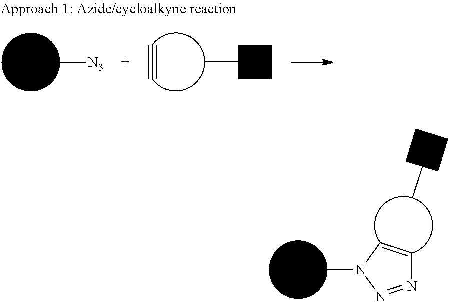

For a review concerning biorthogonal chemistry, including click-chemistry, one can refer to Sletten and Bertozzi, (Angew. Chem. Int. Ed. Engl. 2009, 48(38):6974-6998, the disclosure of which being incorporated herein by reference).

In the context of the present invention, an advantageous free-metal click reaction is strain-promoted alkyne-azide 1,3-dipolar cycloaddition (SPAAC) which refers to the reaction between an azide group and a strained alkyne.

As used herein, a “strained alkyne” refers to a C₆-C₃₀ alkyne wherein the triple bond is sterically strained, in particular a strained cycloalkyne. The strained alkyne may comprise a cyclooctyne scaffold which may be optionally substituted by one or several substituants such as halogens and/or fused to one or several cycles, including heterocycles. For instance, the strained alkyne may comprise one of the following scaffolds:

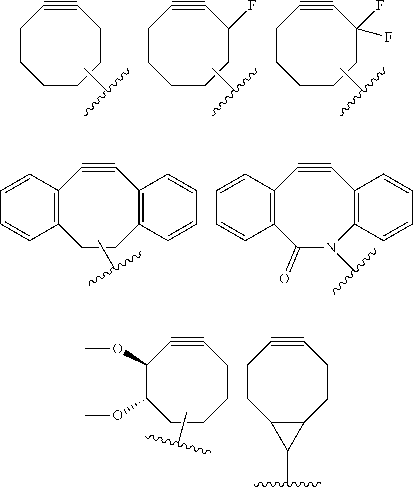

Strained alkynes containing one of said scaffolds can be prepared from commercially available reagents such as OCT, DIBO, BARAC, ALO, DIFO, MOFO, DIBAC and DIMAC:

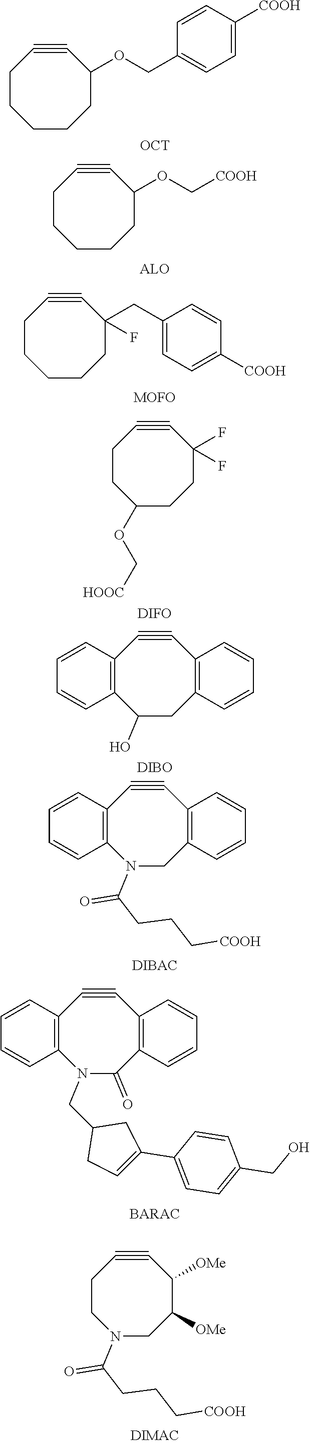

The label bears (or is covalently linked to) an alkyne function (—C≡C), which can be strained or not.

Label can be a directly or indirectly detectable moiety. The label can be selected among dyes, radiolabels and affinity tags. In particular, the dyes can be selected from the group consisting of fluorescent, luminescent or phosphorescent dyes, preferably dansyl, fluorescein, acridine, rhodamine, coumarin, BODIPY and cyanine dyes. More specifically, the fluorescent dyes can be selected among the dyes marketed by Molecular Probes such as the Alexa Fluor dyes, Pacific dyes or Texas Red or by other providers for cyanines 3, 5 and 7. In particular, dyes bearing alkyne, either for CuAAC or SPAAC, are commercially available for Alexa Fluor® 488, 55, 594 and 647 and for TAMRA (tetramethylrhodamine). In a second aspect, the label can be an affinity tag. Such an affinity tag can be for instance selected from the group consisting of biotin, His-tag, Flag-tag, strep-tag, sugars, lipids, sterols, PEG-linkers, and co-factors. In particular embodiment, the label is a biotinylated label, estpecially a biotinylated polyethylene glycol label such as Biotin-PEG4 alkyne (Sigma Aldrich). Biotins linked to alkyne are commercially available, both for CuAAC and SPAAC click chemistry (Biotin DIBO Alkyne by Molecular Probes™). Finally, the label can be a radiolabel. It can be selected from the group consisting of radioactive forms of hydrogen, carbon, phosphorous, sulphur, and iodine, including tritium, carbon-11, carbon-14, phosphorous-32, phosphorous-33, sulphur-33, iodine-123, and iodine-125.

The present invention also relates to a kit comprising a compound according to the present invention and a label bearing an alkyne group or a radical comprising an alkyne group. The alkyne group can be strained or not. Preferably, the label is a fluorescent label or a biotin. The kit may further comprise one or several of the following components: copper (copper(II) precursor with a reducing agent, copper(I) salts or, pre-formed copper(I) complexes); a permeabilizing reagent; a fixation solution; a washing buffer; and a leaflet comprising explanation for the use of the kit.

In a preferred embodiment, the copper reagent is preferably copper(II) with sodium ascorbate. The permeabilizing reagent can be CSK buffer comprising Triton X-100 or any equivalent buffer comprising a detergent suitable for permeabilizing eukaryotic cell membrane. The fixation solution comprises PFA (paraformaldehyde) or any equivalent known by the person skilled in the art. The washing buffer is typically PBS.

The compound of the present invention is useful for labeling DNA-platinated crosslinks in a cell. The method for labeling DNA-platinated crosslinks in a cell comprises a) contacting the cell with a compound of the present invention; and b) contacting said cell with a label bearing an alkyne group, optionally in the presence of copper.

Before step a), the method may comprise an additional step of providing a cell. This step may comprise a step of collecting a sample, e.g., a sample from a patient.

Preferably, between the steps a) and b), the method comprises a step of cell membrane permeabilization, and a step of fixation. More specifically, the method may comprise a step of washing (e.g., for removing free compounds), a step of cell membrane permeabilization, a step of washing, a step of fixation, and then a step of washing. Preferably, these steps are carried out successively in this order, even if the method may optionally comprise additional steps, which can be added between these steps. The inventors observed that performing a permeabilization step before the step of fixation allows to improve the quality and the resolution of the labeling. The purpose of using a permeabilization (e.g., CSK pre-extraction treatment) prior fixation is to remove soluble proteins and RNA loosely bound to chromatin so that the only remaining staining is DNA bound platinum drug analog. By doing so, the resolution of platinum drug analog cross-linked to DNA is higher due to a lower basal level of fluorescence. This enables the detection of foci targeted by platinum drug analog.

Copper or copper precursor is added at step b) if needed depending on the type of alkyne group used in the method.

After the step b), the method may comprise an additional step of washing for removing free label.

Then this method results in the preparation of a cell with DNA-platinated crosslinks covalently bound to the label. The labeled DNA-platinated crosslinks can be used for several goal. This labeling allows the localization, quantification or isolation of DNA-platinated crosslinks.

Indeed, the labeling allows for the detection of DNA-platinated crosslinks in subnuclear regions of the nucleus, thereby allowing to study the localization into the nucleus, and for instance to co-localize with other proteins of interest (such as PCNA, RAD18, DNA polymerases, DNA damage response proteins, DNA repair factors, NER/BER/Fanconi cross links repair factors . . . ) or certain genes of interest.

Accordingly, the present invention also relates to a method for localizing the DNA-platinated crosslinks, the method comprising carrying out the method for labeling DNA-platinated crosslinks in a cell as detailed above, and observing the cell by microscopy, thereby determining the localization of DNA-platinated crosslinks, more particularly their subnuclear localization.

The present invention relates to a method for visualizing platinated DNA crosslinks in cells, the method comprising:
contacting a cell with a compound according to the present invention;
contacting said cell with a label bearing an alkyne group, preferably a fluorescent label, optionally in presence of copper; and
detecting the label in said cell, preferably the fluorescent label.

Preferably, before the step of contacting said cell with a label bearing an alkyne group, the cell is permeabilized and then fixed. Optionally, washing steps are carried out when necessary.

In an alternative method, it can be contemplated to permeablize and/or fix the cells before contacting them with the compound according to the present invention.

In addition, the labeling of the DNA-platinated crosslinks authorizes the quantification of the number of DNA-platinated crosslinks. For instance, if the label is fluorescent, the amount of fluorescence can be measured, this amount being proportional to the amount of DNA-platinated crosslinks. If the label is radioactive, then the amount of radioactivity is measured. In a preferred embodiment, the label is fluorescent. Therefore, the present invention relates to a method for quantifying the number of DNA-platinated crosslinks, the method comprising carrying out the method for labeling DNA-platinated crosslinks in a cell as detailed above, and measuring the signal emitted by the label. More particularly, if the label is fluorescent, the signal is the emitted fluorescence. In addition or alternatively, the present invention relates to a method for localizing the DNA-platinated crosslinks in a cell. In this embodiment, the method comprises carrying out the method for labeling DNA-platinated crosslinks in a cell as detailed above, and localizing the DNA-platinated crosslinks. In addition, other proteins or nucleic acid sequences can be further labeled and the localization of the labelings can be compared, for instance to study co-localization.

The sensitivity of a cell to a platinum drug is generally proportional to the number of DNA-platinated crosslinks. By sensitivity is intended to refer to the capacity of the platinum drug to kill the cell, by apoptosis or any other killing process. Accordingly, the sensitivity of a cell to a platinum drug is then proportional to the intensity of the label signal, e.g., the fluorescence amount. Then, higher is the intensity of the label signal, better will be the sensitivity of the cell to a platinum drug. Inversely, lower is the intensity of the label signal, lower will be the sensitivity of the cell to a platinum drug or higher will be the likelihood of a resistance to a platinum drug treatment. The intensity of the label signal can be compared to a reference intensity of the label signal. For instance, the reference intensity of the signal is the intensity measured in a cell known for being sensitive to a platinum drug. Alternatively, the reference intensity of the signal is the intensity measured in a cell known for being resistant to a platinum drug. Preferably, the cell of reference is the closest of the cell to be studied.

Therefore, the present invention relates to the use of a compound or a kit of the present invention for predicting a sensitivity or resistance to a platinum drug in a patient. More particularly, it relates to a method for predicting a resistance or sensitivity of a tumor in a patient to a platinum drug, comprising
carrying out the method for visualizing platinated DNA crosslinks as detailed above with a cell from a tumor sample from the patient;
measuring the labeling and optionally comparing the labeling to a reference level; and
determining the resistance or sensitivity to a platinum drug of the tumor in the patient based on the intensity of the labeling, the sensitivity being proportional to the intensity of the labeling.

As detailed above, the reference level can be the intensity measured in a cell known for being sensitive to a platinum drug and/or the intensity measured in a cell known for being resistant to a platinum drug. Preferably, the cell of reference is the closest of the cell to be studied. Alternatively, the reference level can be the level measured in a cell from the same patient, preferably a non-cancerous cell, for instance a corresponding histological normal reference tissue, in particular from the vicinity of the tumour. Then the method may comprise a previous step of providing a tumor sample and a histologically matched normal tissue from the patient.

In another alternative, the reference cell can be a tumor cell from the same patient but before or at the beginning of the treatment by a platinum drug. Then, in this aspect, the method can be used for following the occurrence of a resistance to a platinum drug in a patient.

Alternatively or in addition, the present invention relates to a method for predicting a resistance or sensitivity of a tumor in a patient to a platinum drug, comprising
carrying out the method for visualizing platinated DNA crosslinks as detailed above with a cell from a tumor sample from the patient;
determining the localization of the labeling and optionally comparing the localization of the labeling to a reference localization; and
determining the resistance or sensitivity to a platinum drug of the tumor in the patient based on the localization of the labeling.

A resistance or sensitivity to a platinum drug can be determined based on a change of localization of the labeling.

In addition, the present invention relates to the use of a compound or a kit of the present invention for identifying or screening a molecule capable of preventing or delaying the occurrence of resistance to platinum drugs or to overcome or reduce resistance to platinum drugs. Indeed, as the present application discloses a mean for evaluating the sensitivity or resistance of a cell to a platinum drug which can be implemented at a high throughput level, it can be then used in a method suitable for testing a library of molecules. The present invention relates to a method for identifying or screening a molecule capable of preventing or delaying the occurrence of resistance to platinum drugs or to overcome or reduce resistance to platinum drugs, the method comprising:
contacting a cell with a compound according to the present invention and with a candidate molecule, wherein the contact with the compound can be after, simultaneously, or before the contact with the candidate molecule;
contacting said cell with a label bearing an alkyne group, preferably a fluorescent label, in presence of copper;
measuring the labeling;
optionally comparing the intensity of the labeling in the presence and the absence of the candidate molecule;
selecting the candidate molecule if the intensity of the labeling is increased in presence of the candidate molecule when compared to the intensity of the labeling in absence of candidate molecule.

In a preferred embodiment, the cell is a cell which is resistant to a platinum drug.

Alternatively or in addition, the impact of the candidate molecule on localization of the labeling can be considered as a marker of the sensitivity or resistance to the platinum drug. Therefore, the impact of the candidate molecule on the morphology of foci can also be studied.

In a preferred embodiment, the compound of the present invention is contacted with the cell after its incubation in presence of the candidate molecule. The cell is incubated with the candidate molecule during a period from 1 hour to 5 days, preferably form 1 day to 4 days, for instance 3 days. Alternatively, the cell can be incubated simultaneously with the compound of the invention and the candidate molecule. In a last embodiment, the cell is incubated with the compound of the invention before the addition of the candidate molecule. Optionally, washing step can be added when necessary.

Optionally, the effect of candidate molecule can be compared with molecules already known to have an effect on the sensitivity of cell to a platinum drug, for instance a histone deacetylase inhibitor. Optionally, a combination of candidate molecules can be also tested by the present screening method.

Preferably, the cells used in the methods of the present invention are cancer cells. It can provide from a cancer cell line or a cell from primary tumors. It can be resistant to a platinum drug, more specifically resistant to cisplatin. Cell lines resistant to a platinum drug are commercially available (ATCC). Preferably, the cells are mammalian cells, and more specifically human cells. Non-exhaustive examples of suitable cells include ovarian cells such as A2780 and A2780cisR cells such as OV2008, CaoV-3, OVCAR-3, SKOV-3, PEA1/A2, PEO14/23, PEO1/4/6, IGROV-1, non-small-cell lung cancer cells such as A549 and H292, breast cancer cells such as MBA-MD-231, osteosarcoma cells such as U2OS, colon cells such as HCT-116.

Finally, the present invention also relates to the use of a compound or a kit of the present invention for isolating DNA-platinated crosslinks, more specifically isolating the DNA sequences comprising DNA-platinated crosslinks (pull-down methodology). Indeed, the present invention authorizes the high throughput sequencing of the isolated sequences. The general strategy is described in the FIG. 3E. The present invention relates to a method comprising a) contacting a cell with a compound of the present invention, b) purifying or isolating the genomic DNA from the cell, c) adding an affinity tag bearing an alkyne group, optionally in presence of copper if necessary; d) isolating or purifying the genomic DNA linked to the affinity tag. Optionally, the method may comprise a step of removing RNA, in particular during step b). Optionally, the method may comprise before step d) a step of fragmenting DNA. Preferably, step d) is carried out by contacting the DNA with a solid support on which a molecule able to bind the affinity tag has been immobilized, for instance beads. Preferably, the method comprises an additional step after step d) of reversing DNA-platinated crosslinks, for instance by using thiourea.

Preferably, the affinity tag is a biotin. Biotins linked to alkyne are commercially available, both for CuAAC and SPAAC click chemistry (Biotin-PEG4 alkyne by Sigma Aldrich; or Biotin DIBO Alkyne by Molecular Probes™). Then, streptavidin can be used in step d) for isolating or purifying the genomic DNA linked to the biotin. Of course, the method can be easily adapted with another couple of affinity tag-binding agent.

The recovered DNA can be used by the person skilled in the art for any kind of analysis. In particular, this recovered DNA can be sequenced.

Further aspects and advantages of the present invention will be described in the following examples, which should be regarded as illustrative and not limiting.

### BRIEF DESCRIPTION OF THE FIGURES

FIG. 1|Design, synthesis and validation of ACP as a clickable cisplatin probe. (FIG. 1a) Molecular structures of platinum drugs. (FIG. 1b) Synthetic route to ACP. (i) (COCl)2, DCM, cat. DMF, rt, 2 h. (ii) MeOH, rt, 16 h, 74%. (iii) CaCl2), NaBH4, MeOH:THF (1:1), 0° C. to rt, 48 h, 87%. (iv) NaN3, DMF:H2O (20:1), 85° C., 14 d, 60%. (v) SOCl2, CHCl3, 0° C. to rt, 16 h, 70%. (vi) Potassium phthalimide, DMF, rt, 16 h, 93%. (vii) NH2NH2.H2O, THF:MeOH (1:1), rt, 16 h, 36%. (viii) K2PtCl4, H2O, rt, 3 h, 54%. (FIG. 1c) Anti-proliferative activity of platinum drugs against U2OS cells. (FIG. 1d) Schematic representation of a DNA hairpin (hp), a 1,2-GG intra-strand platinum DNA crosslink (hp-Pt) and chemical labeling of the platinated DNA lesion using click labeling (hp-Pt-488). For clarity, only a single regioisomer is shown for hp-Pt-488. (FIG. 1e) Mass spectrometry detection of a free DNA hairpin, the corresponding platinated DNA adduct and its labeled counterpart. hp-Pt was observed as the molecular ion peak with azide fragmentation. (FIG. 1f) Detection of genomic DNA platination from U2OS cells by dot blot.

FIG. 2|Cellular localization of DNA-Pt. (a) Fluorescence labeling by click chemistry and localization of DNA-Pt in U2OS cells. (b) Schematic representation of a strategy for enhancing the detection of DNA-Pt in cells. (c) Visual detection by fluorescence microscopy of labeled DNA-Pt in U2OS cells subjected to pre-extraction. Zoomed images are ×4. Scale bar, 10 μm.

FIG. 3|Unbiased screening identifies HDAC inhibition as a regulator of genome targeting with a platinum drug. (a) Small molecules screened for modulation of cisplatin targeting. (b) Visual detection of labeled DNA-Pt in U2OS cells treated as indicated. White arrows indicate nucleolar targeting. Scale bar, 20 μm. (c) Quantification of b. (d) Western blot analysis of osteosarcoma U2OS cells treated as indicated showing hyperacetylation of H4. (e) Schematic representation of platinum-bound DNA pull-down methodology. (f) Quantification of DNA recovered by pull-down from samples as indicated. Error bars represent SEM (N=3).

FIG. 4|HDAC inhibition sensitizes cancer cells to platinum drugs by promoting TLS and apoptosis. (a) Activation of TLS by SAHA/ACP treatment. Western blot analysis of PCNA mono-ubiquitination in U2OS cells treated as indicated. (b) Co-localization of DNA-Pt with RAD18 in U2OS cells treated as indicated. Zoomed images are ×3. Scale bar, 20 μm. (c) and (d) Western blot analysis of PCNA mono-ubiquitination and apoptotic markers in WT and RAD18 KO HCT-116 cells treated as indicated.

FIG. 5|Comparative analysis of APPA and APPOA by visual detection with fluorescence microscopy of labeled DNA-Pt in U2OS cells subjected to pre-extraction. Zoomed images are ×3. Scale bar, 20 μm.

### EXAMPLES

To study cisplatin lesions, the inventors sought to develop a surrogate probe that would allow for the chemical labeling of target-bound platinum in cells post drug treatment. The ability to visually detect DNA-Pt at the single-cell level would provide the means to monitor proteins at sites of lesions and to identify small molecules with a propensity to modulate targeting with cisplatin in an unbiased manner. In addition, the pull down is also a robust technique to compare isolated platinum bound DNA between responsive and resistant cell line

### Results

### Chemical Labeling of a Platinum Drug in Cells.

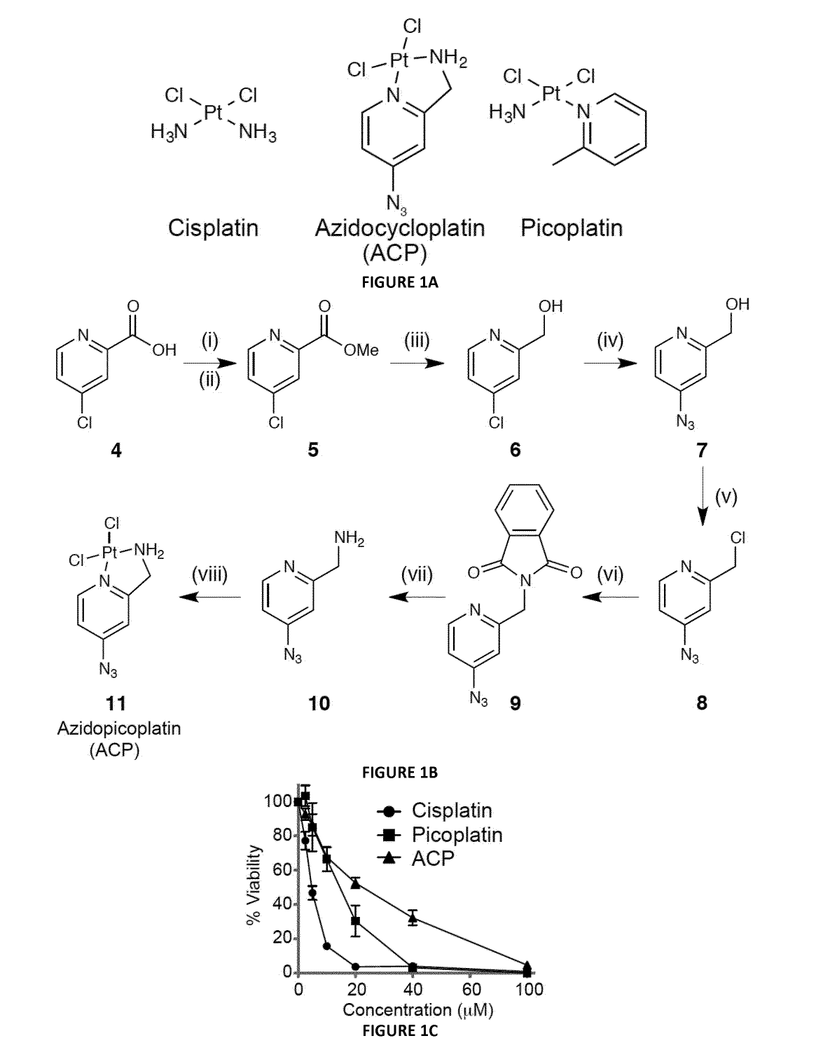

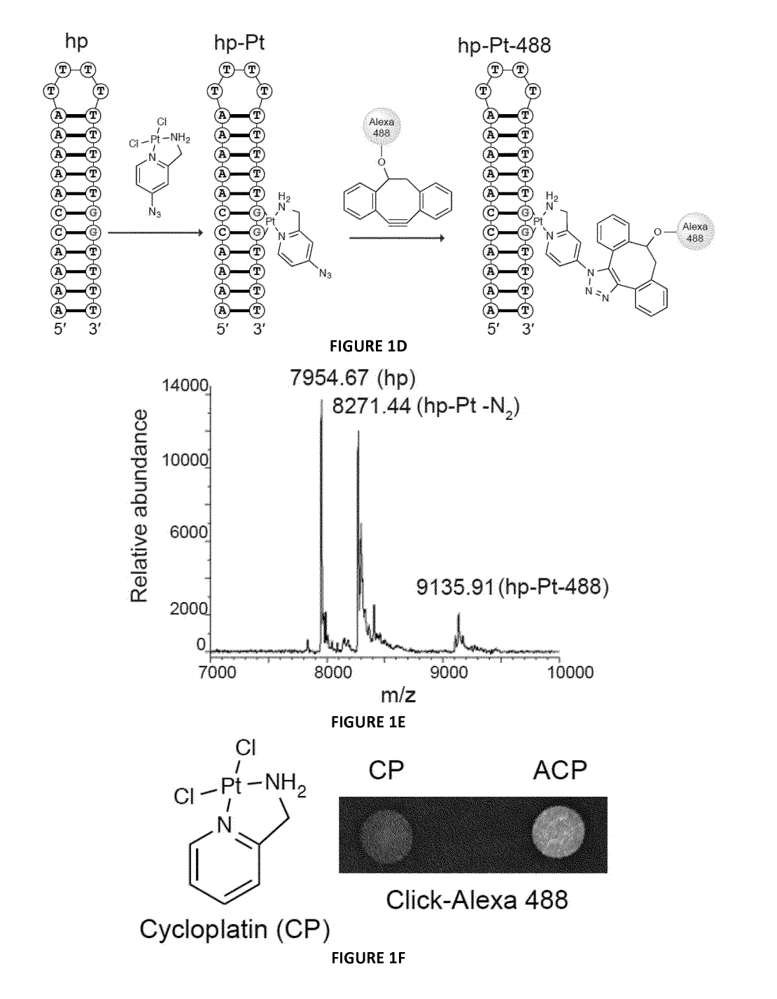

To study cisplatin lesions, the inventors sought to develop a surrogate probe that would allow for the chemical labeling of DNA-Pt crosslinks in cells. Prior expertise in elucidating mechanisms of action of small molecules prompted us to develop an azide-containing drug to label platinated DNA adducts by means of bio-orthogonal click chemistry. Here, a significant challenge consisted of functionalizing the inorganic platinum substrate with an organic moiety without altering the reactivity of the metal towards DNA and to maintain acceptable biological activity. The inventors synthesized the cyclic azidoplatinum-containing drug named azidocycloplatin (ACP, FIG. 1a and 1b). The design of ACP was inspired from the structure of picoplatin (FIG. 1a), taking advantage of the aromatic methyl substituent to form a rigid five-membered ring with Pt. Thus, ACP exhibits a structure reminiscent of that of oxaliplatin, where the ring prevents free rotation of the pyridine core chelated to platinum. This structural distinction is not trivial given that the processing of DNA-Pt in cells heavily relies on the stability, size and dynamics of these lesions. The synthetic route based on the formation of a cyclic platinum adduct was also devoid of silver reagents, making the synthesis tractable and leading to pure compound suitable for biological evaluation.

Like cisplatin and picoplatin, ACP exhibited anti-proliferative properties in human osteosarcoma U2OS cells (FIG. 1c). The inventors next evaluated the reactivity of ACP towards DNA and the ability to label DNA-Pt in vitro and in cells with a complementary alkyne-containing fluorophore by means of click chemistry. A 26-mer hairpin-forming DNA oligonucleotide containing a single 1,2-GG dinucleotide, which is prone to forming intra-strand crosslinks with platinum drugs, was incubated with ACP, purified and then reacted with a strained alkyne-containing Alexa 488 (FIG. 1d) (Baskin et al, 2007, Proc Natl Acad Sci USA, 104, 16793-16797; Jewett et al, 2010, Chem Soc Rev, 39, 1272-1279). The reaction products were then analyzed and characterized by mass spectrometry using Matrix-Assisted Laser Desorption/Ionization (MALDI) analysis. The inventors identified three ion peaks corresponding to the free unreacted hairpin along with the unlabeled and fluorescently labeled ACP adducts (FIG. 1e). These results demonstrated that the bi-functional ACP can chemoselectively cross react with DNA and be labeled by an alkyne-containing tag in vitro sequentially. The inventors next performed similar experiments directly in cells using ACP and the control compound cycloplatin (CP, FIG. 1f), a structurally related active analogue of ACP devoid of azide functionality and therefore not amenable to click chemistry. Labeled genomic DNA obtained from ACP-treated cells displayed a higher level of fluorescence compared to equal amounts of DNA collected from CP-treated cells as monitored by dot blot (FIG. 1f). Taken together, these data demonstrated that ACP reacted with DNA and that DNA-Pt crosslinks can be labeled in vitro and in cells.

### Cellular Localization of DNA-Pt Crosslinks.

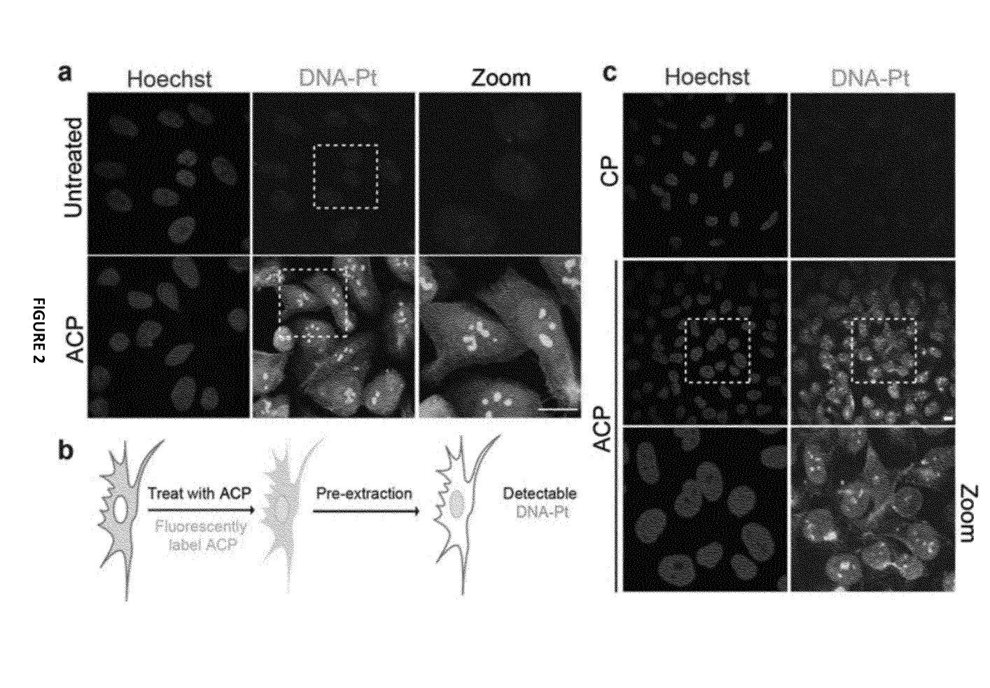

With the successful development and validation of this probe, the inventors next sought to further elaborate on this technology to evaluate the localization of DNA-Pt in cells. To this end, cells were treated with ACP and fixed with formaldehyde prior to being subjected to copper catalysis to label DNA lesions. Labeled drug adducts exhibited a diffuse cytoplasmic and nuclear staining (FIG. 2a), which was consistent with previous observations for the reported distribution of cisplatin derivatives in cells (Ding et al, supra; Liang et al, 2005, J Cell Physiol, 202, 635-641; Qiao et al, 2014, Journal of biological inorganic chemistry:JBIC:a publication of the Society of Biological Inorganic Chemistry 19, 415-426). As a means to selectively detect DNA-drug adducts, the inventors developed a pre-extraction protocol to remove nuclear proteins and RNA substrates of platinum drugs associated to chromatin that would otherwise preclude the identification of these lesions with the required resolution (FIG. 2b). This protocol allowed for the detection of DNA-Pt in the nucleus with some subnuclear regions displaying increased fluorescence intensity (FIG. 2c). Interestingly, labeled DNA-Pt lesions co-localized with the nucleolar protein fibrillarin, in agreement with the idea that platinum drugs target rRNA. Collectively, these data validated ACP as a functional clickable cisplatin probe with which to study genome targeting and responses to platinum drugs.

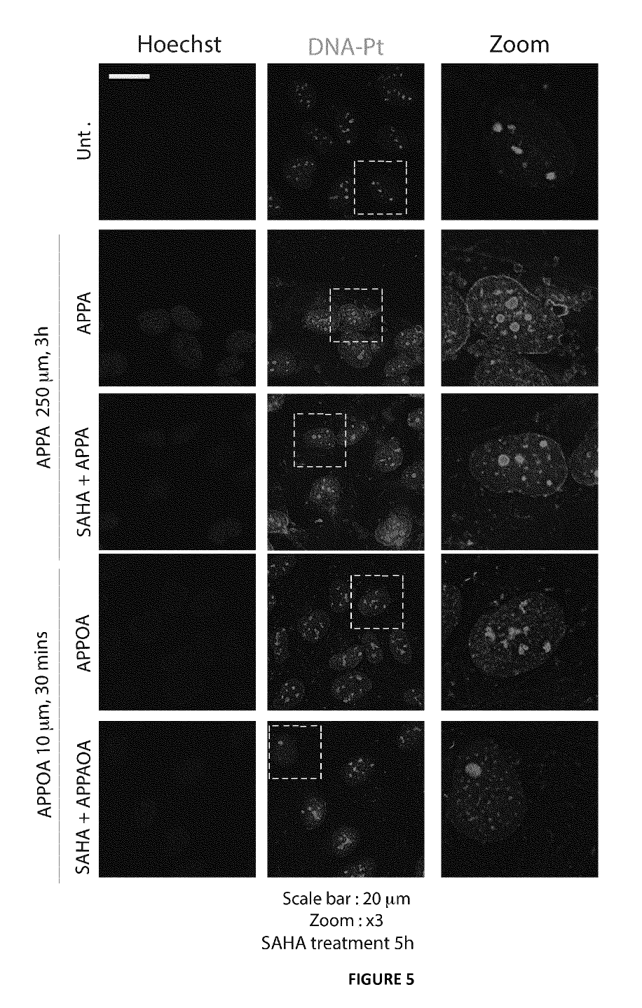

In addition, the inventors further tested APPOA and the results are shown in FIG. 5.

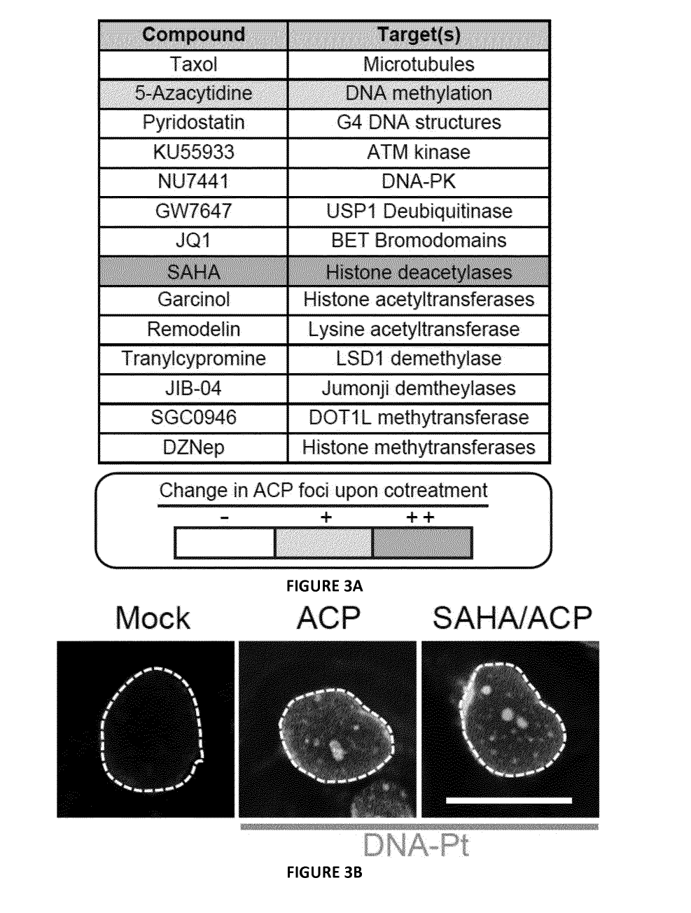

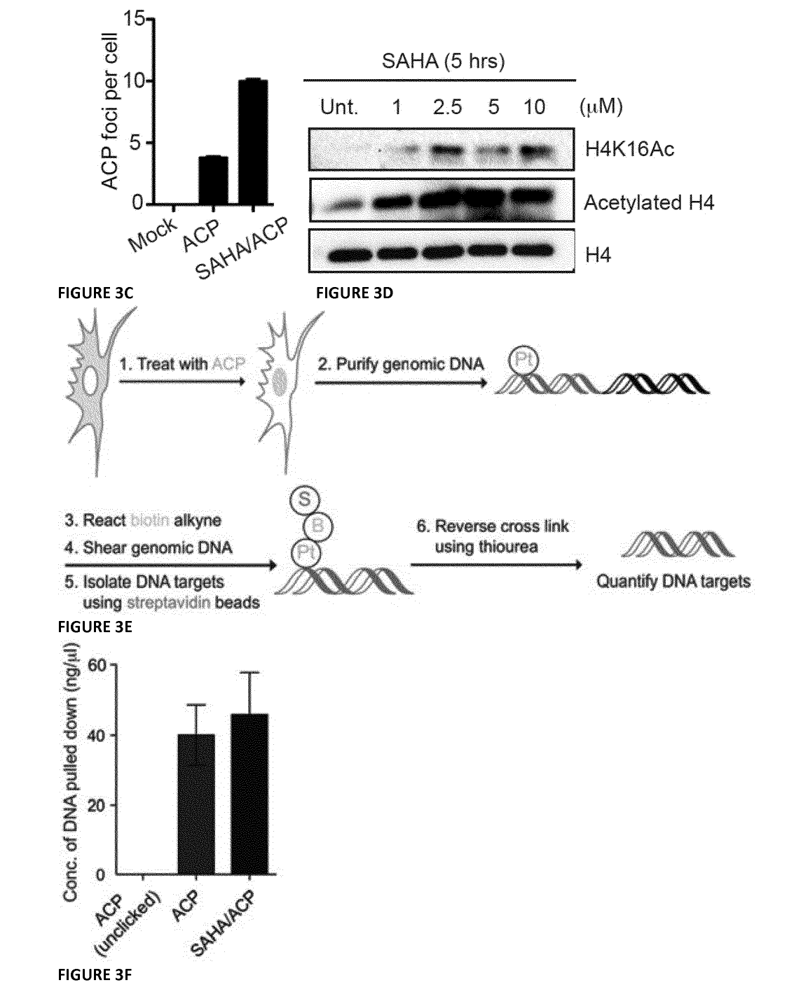

With this optimized technology in hand, the inventors next searched for small molecule modulators of genomic targeting with cisplatin using ACP staining as a readout. Thus, they screened a defined set of small molecules operating at the level of chromatin or that are used in cancer treatments in conjunction with cisplatin (FIG. 3a, Table 1).

TABLE 1

| Small molecule | Biological target/phenotype | Time of treatment | |
|---|---|---|---|
| Taxol | β-tubulin/stabilizes | 6 | h |
| | microtubules | | |
| 5-Azacytidine | DNMT1 and DNMT3/induce | 24 | h |
| | DNA hypomethylation | | |
| Pyridostatin | G-quadruplex motif/alters | 6 | h |
| | gene transcription | | |
| KU55933 | ATM/disrupts the signaling | 4 | h |
| | and repair of DSBs | | |
| NU7441 | DNA-PK/disrupts the | 4 | h |
| | signaling and repair DSBs | | |
| GW7647 | USP1/enhancing TLS and | 3 | h |
| | Fanconi anemia activity | | |
| JQ1 | BET bromodomains/inhibits | 5 | h |
| | BET-dependent | | |
| | transcription | | |
| SAHA | HDACs/induces chromatin | 5 | h |
| | relaxation | | |
| Garcinol | p300 and PCAF (HATs) | 24 | h |
| Remodelin | NAT10/alters microtubule | 24 | h |
| | nucleation | | |
| Tranylcypromine | LSD1/BHC110 | 12 and 24 | h |
| JIB-04 | Pan Jumonji HDMTs | 24 | h |
| | inhibitor | | |
| SGC0946 | DOT1L (HMT) | 24 and 48 | h |
| DZNep | Pan HMTs inhibitor | 24 | h |

U2OS cells were co-treated with each small molecule independently and ACP, then subjected to click-labeling. Labeled DNA-Pt were analyzed by confocal microscopy. While most small molecules had no discernable effect on ACP staining by visual inspection, pre-treatment with the clinically approved drugs 5-Aza (Christman, J. K., 2002, Oncogene 21, 5483-5495) and Vorinostat (SAHA) (Marks, P. A. & Breslow, R., 2007, Nature biotechnology 25, 84-90) led to the occurrence of foci of DNA-Pt, indicating the presence of clusters of purine-residues at these sites (FIGS. 3b and c). These data were consistent with the notion that chromatin relaxation resulting from SAHA treatment revealed de novo DNA targets of ACP. Indeed, the inventors confirmed that SAHA induced histone hyperacetylation of histone H4, a well-established marker of open chromatin (FIG. 3d). It is noteworthy that ACP lesions occurring in SAHA-treated cells did not co-localize with CENPA (i.e. centromeres) or TRF1 (i.e. telomeres), excluding these loci containing repetitive sequences rich in 1,2-purine residues as primary ACP targets. As a control, RNA-Seq analysis identified a small subset of genes that were up- or down-regulated by ACP, which remained mostly unaffected by SAHA, supporting the idea that increased ACP loading by SAHA occurred independently of a general transcriptional alteration in response to the drug. To substantiate this result, the inventors developed a protocol to isolate DNA targets of ACP from cells (FIG. 3e). Cells were either treated with ACP- or SAHA/ACP and subjected to affinity pull-down as previously reported by us for other small molecules (Rodriguez, R. & Miller, K. M. Nature reviews. Genetics 15, 783-796 (2014); Rodriguez, R. et al. Nature chemical biology 8, 301-310 (2012); Larrieu, D., et al. Science 344, 527-532 (2014)). The amount of DNA pulled down from ACP- and SAHA/ACP-treated cells was statistically similar, which was in line with the idea that SAHA does not solely act by increasing the number of DNA targets per se, but rather potentiates genome targeting with platinum at particular sites (FIG. 3f). These data supported the notion that chromatin relaxation increased genome accessibility to ACP and provided additional insights into how SAHA sensitizes cells to genotoxic compounds including cisplatin.

Turning Translesion Synthesis (TLS) into an Apoptotic Trigger.

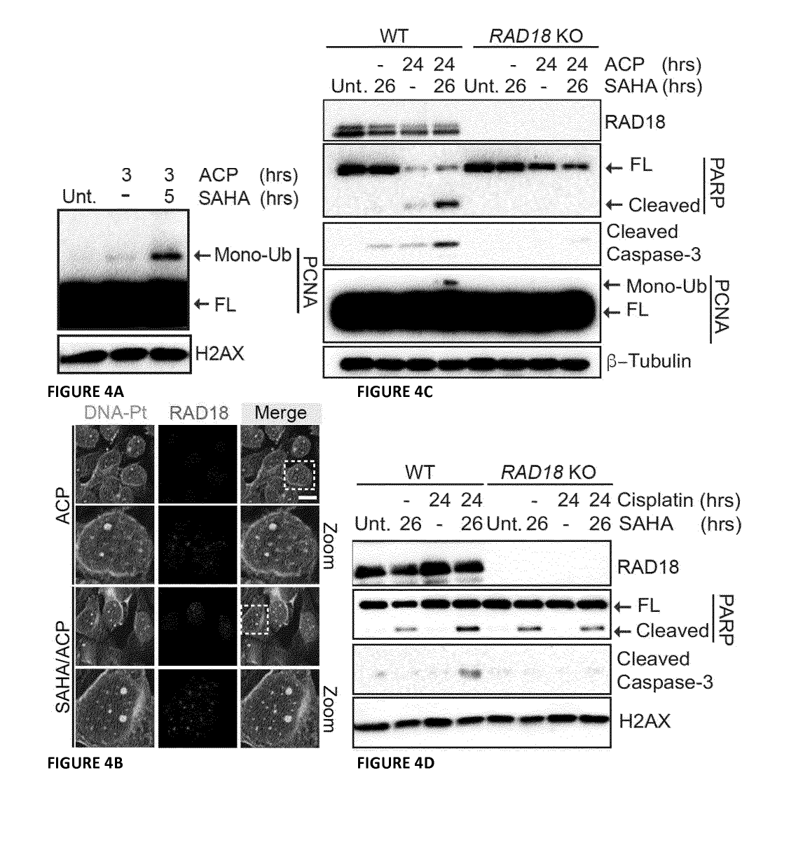

The presence of ACP foci upon SAHA treatment prompted the inventors to determine whether clusters of DNA-Pt could act as replication roadblocks requiring TLS to bypass these lesions. TLS activation was readily detected in U2OS cells co-treated with SAHA/ACP as defined by the mono-ubiquitination of proliferating cell nuclear antigen (PCNA; FIG. 4a), a key marker of TLS. Remarkably, foci of DNA-Pt co-localized with the TLS factor RAD18, a E3 Ubiquitin ligase that mediates mono-ubiquitination of PCNA (FIG. 4b). Strikingly, co-treatment with SAHA and ACP induced higher levels of DNA damage (i.e. γH2AX) and apoptosis as defined by the cleavage of PARP and the activation of caspase 3, respectively, in several cancer cell lines including colon HCT116, osteosarcoma U2OS, and ovarian A2780 (FIG. 4c). These data indicated that DNA-Pt resulting from HDAC inhibition activated TLS and apoptosis. To evaluate whether TLS was directly involved in apoptotic signaling under these conditions, the inventors performed similar experiments with matched HCT116 RAD18 knockout (KO) cells. Western blotting indicated that cells devoid of RAD18 did not display PCNA mono-ubiquitination in response to SAHA/ACP treatment while it was observed in WT cells (FIG. 4c). Interestingly, markers of apoptosis were not detected in HCT-116 RAD18 KO cells even though ACP foci formed similarly in WT and RAD18 KO cells co-treated with SAHA and ACP (FIG. 4c). These results confirmed the RAD18-dependent PCNA mono-ubiquitination in these cells and implicated TLS in initiating apoptosis in response to SAHA and ACP. Similar results were observed in cisplatin-treated WT and RAD18 KO cells, demonstrating a general response to these drugs (FIG. 4d). Although the promotion of apoptosis by TLS could be potentially counterintuitive owing to the well-established role of this machinery in resistance to cisplatin, HDAC inhibition has been shown to re-sensitize resistant cancer cells to this drug. The present results suggest that the higher level of apoptosis signaling in RAD18 expressing cells in response to platinum drugs and SAHA treatment is due to the inability of TLS to efficiently bypass DNA-Pt clusters. These results are directly attributable to the capacity to visually detect DNA-Pt crosslinks and associated proteins with high resolution.

### Discussion

The inventors have developed a versatile strategy based on a novel cisplatin analogue and a pre-extraction protocol, which enabled the unbiased identification of small molecule modulators of genome targeting with cisplatin and the direct visualization of TLS activation at sites of DNA-Pt crosslinks. Engagement of the replication machinery with cisplatin lesions results in fork stalling and collapse, processes that promote genome instability and cell death. However, cells can employ a DNA damage tolerance pathway involving the recruitment of specialized low fidelity polymerases to mono-ubiquitinated PCNA allowing for lesion bypass. The aptitude to tolerate these lesions through this pathway has been shown to play a critical role in resistance to cisplatin, a significant impediment for the use of these drugs in the clinic. To overcome these limitations, cisplatin analogs containing bulkier ligands or combination therapies with other drugs have been studied. For example, co-administration of histone deacetylase or DNA methylation inhibitors sensitize cancer cells to DNA-damaging agents and HDAC inhibition has been shown to resensitize resistant cancer cells to cisplatin. The inventors discovered that treating cells with the cisplatin analog ACP and SAHA resulted in TLS activation at sites of DNA-Pt as confirmed by increased PCNA ubiquitination and RAD18 localization at these sites (FIG. 4). Interestingly, this treatment did not enable TLS to bypass de novo platinated lesions, triggering instead TLS-dependent apoptosis. Thus, these data demonstrate that altered genome targeting of platinum drugs through chromatin remodeling inhibits the active process of translesion synthesis and suggest retooling of TLS function in this context. These findings provide new insights into how chromatin alterations can circumvent intrinsic and acquired resistance of cancer cells to certain drugs, findings that can be exploited and further developed for the clinical management of cancer. This work has raised several new questions that merit further investigation including the characterization of these particular lesions able to circumvent TLS bypass and defining how these lesions trigger programmed cell death.

The present data is consistent with a model whereby chromatin can alter the accessibility of the genome to small molecules, which impacts the cellular response to these drugs. Genome and epigenome targeting drugs represent a large class of compounds used as therapeutics and molecular biology reagents. The methodology described here has delivered unanticipated insights into how chromatin remodeling sensitizes cancer cells to cisplatin, establishing a powerful experimental platform for basic and translational research relying on small molecules.

### Materials & Methods

### Synthesis

All starting materials were purchased from commercial sources and used without further purification, or purified according to Purification of Laboratory Chemicals (Armarego, W. L. F., Chai, C. L. L. 5^{th} edition). Solvents were dried under standard conditions. Reactions were monitored by thin-layer chromatography (TLC) using TLC silica gel coated aluminum plates 60E-254 (Merck). Column chromatography was performed using Merck silica gel 60, 0.040-0.063 mm (230-400 mesh). NMR spectroscopy was performed on Bruker 300, 500 MHz apparatus equipped with a cryoprobe. Spectra were run in CDCl₃, or DME-d₇ at 298 K unless otherwise stated. Molecular structures have been characterized using a comprehensive dataset including ¹H- and ¹³C-NMR spectra (1D and 2D experiments). ¹H chemical shifts are expressed in ppm using the residual non deuterated solvents as internal standard (CDCl₃ ¹H, 7.26 ppm) and (DME-d₇ ¹H, 8.03, 2.92, 2.75 ppm). The following abbreviations are used: s, singlet; d, doublet; dd, double doublet; t, triplet; td, triplet doublet; q, quartet; m, multiplet; bs, broad singlet. ¹³C chemical shifts are expressed in ppm using the residual non deuterated solvents as internal standard (CDCl₃ ¹³C, 77.16 ppm) and (DMF-d₇ ¹³C, 163.15, 34.89, 29.76 ppm). Exact masses were recorded on a LCT Premier XE (Waters) equipped with an ESI ionization source and a TOF detector and on a Q-TOF 6540 (Agilent).

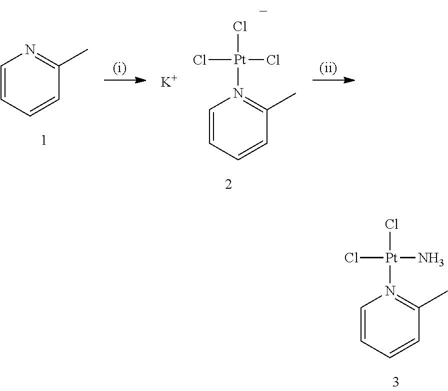

### Synthesis of Picoplatin. (i)

K2PtCl4, NMP, 60° C., 4 h, 63%. (ii) KCl (2.5 N), CH3CO₂NH₄, NH₄OH (2.5 N), 45° C., 1 h, 49%.

### K[PtCl₃(2-picoline)] (2)

Compound 2 was prepared according to a previously published procedure (U.S. Pat. No. 6,413,953). To a suspension of K₂PtCl₄ (300 mg, 0.72 mmol) in N-methyl-2-pyrrolidone (1.2 ml) was added a solution of commercially available 2-picoline 1 (74 mg, 0.79 mmol) in N-methyl-2-pyrrolidone (0.9 ml) portionwise. The rate of the addition was 20% of the solution per 30 min. After addition of the first portion, the reaction mixture was immersed in an oil bath and stirred at 60° C. for 4 h. Then, the mixture was allowed to reach room temperature, followed by addition of dichloromethane (9 ml). The precipitants KCl and K[PtCl₃(2-picoline)] were collected by filtration and washed with dichloromethane (3×1 ml) and diethyl ether (3×1 ml). The product was dried under reduced pressure to afford 2 and KCl (250 mg, 63%) as a yellow solid. ¹H NMR (500 MHz, DME-d₇): δ 8.99 (d, J=6.0 Hz, 1H), 7.72 (t, J=7.5 Hz, 1H), 7.42 (d, J=7.5 Hz, 1H), 7.22 (t, J=6.0 Hz, 1H), 3.24 (s, 3H).

### Picoplatin (3)

Compound 3 was prepared according to a previously published procedure (U.S. Pat. No. 6,413,953). To a solution of K[PtCl₃(2-picoline)]/KCl (231 mg, 0.42 mmol) dissolved in a KCl solution (0.33 ml, 2.5 N) was added ammonium acetate (163 mg, 2.12 mmol) diluted in an ammonium hydroxide solution (0.84 ml, 2.5 N). The resulting mixture was stirred in the dark at 45° C. for 1 h. The precipitate was collected by filtration and was washed with water (2×1 ml) and acetone (2×1 ml). The product was dried under reduced pressure to afford 3 (78 mg, 49%) as a yellow solid. ¹H NMR (500 MHz, DME-d₇): δ 9.02 (d, J=6.0 Hz, 1H), 7.86 (t, J=7.5 Hz, 1H), 7.54 (d, J=7.5 Hz, 1H), 7.34 (t, J=6.0 Hz, 1H), 4.39 (br s, 3H), 3.18 (s, 3H). HRMS (ESI-TOF) calcd. for C₆H₁₀Cl₂N₂NaPt⁺ [M+Na]⁺ 397.9766, found: 398.9744.

### Methyl 4-chloropicolinate (5)

Compound 5 was prepared according to a modified procedure (WO2013/057253). To a suspension of the commercially available 4-chloro-pyridine-2-carboxylic acid 4 (5.0 g, 31.84 mmol) in dichloromethane (135 ml) at 0° C. was added oxalyl chloride (4.8 g, 38.21 mmol), followed by a slow addition of catalytic amount of dimethylformamide (0.55 ml). The resulting mixture was stirred at room temperature for 2 h. After this time, the mixture was concentrated to dryness under reduced pressure. The solid residue was solubilized in methanol (55 ml) and was stirred at room temperature for another 16 h. The mixture was concentrated to dryness under reduced pressure, and the residue re-suspended with 5% aq. NaHCO₃. The product was extracted with EtOAc (2×20 ml). The combined organic layer was washed with brine (2×10 ml), dried over anhydrous MgSO₄, filtered and concentrated to dryness under reduced pressure to afford 5 (4.0 g, 74%) as a beige solid. ¹H NMR (300 MHz, CDCl₃): δ 8.63 (d, J=5.0 Hz, 1H), 8.12 (dd, J=2.0, 0.5 Hz, 1H), 7.48 (dd, J=5.0, 2.0 Hz, 1H), 4.00 (s, 3H). ¹³C NMR (75 MHz, CDCl₃): δ 164.7, 150.7, 149.3, 145.5, 127.2, 125.7, 53.3. HRMS (APPI) calcd. for C₇H₇ClNO₂⁺[M+H]⁺ 172.0160, found: 172.0156.

### (4-Chloropyridin-2-yl)methanol (6)

Compound 6 was prepared according to a modified procedure (Comba, P., et al. Inorg. Chem. 52, 6481-6501 (2013)). To a mixture of methanol (24 ml) and tetrahydrofurane (14 ml) were added 5 (4.1 g, 23.87 mmol) and calcium chloride (10.5 g, 95.48 mmol). The reaction mixture was cooled to 0° C. Then, sodium borohydride (1.8 g, 47.74 mmol) was added portionwise. The resulting mixture was stirred at room temperature for 24 h. Then, the same amounts of methanol, tetrahydrofurane, calcium chloride, and sodium borohydride were added following the same procedure, and the reaction mixture was stirred for 24 h. After this time, water (80 ml) was added to the reaction mixture, which was stirred for 2 h. The product was extracted with EtOAc (3×180 ml). The combined organic layer was washed with brine (100 ml), dried over MgSO₄ and concentrated to dryness under reduced pressure to afford 6 (3.0 g, 87%) as a pale white solid. ¹H NMR (300 MHz, CDCl₃): δ 8.38 (d, J=5.5 Hz, 1H), 7.34 (s, 1H), 7.18 (dd, J=5.5, 2.0 Hz, 1H), 4.71 (s, 2H), 4.19 (br s, 1H). ¹³C NMR (75 MHz, CDCl₃): δ 161.6, 149.5, 145.0, 122.8, 121.1, 64.2. HRMS (ESI-TOF) calcd. for C₆H₆ClNNaO⁺ [M+Na]⁺ 166.0036, found: 166.0028.

### (4-Azidopyridin-2-yl)methanol (7)

Sodium azide (1.3 g, 20.89 mmol) was added to a mixture of 6 (1.0 g, 6.96 mmol) in dimethylformamide (10.5 ml) and water (0.52 ml). The resulting mixture was stirred at 85° C. for 14 d. After this time, water (5 ml) was added and the product was extracted with EtOAc (3×7 ml). The combined organic layer was washed with brine (7 ml), dried over anhydrous MgSO₄, filtered and concentrated to dryness under reduced pressure to afford 7 (634 mg, 60%) as a pale yellow solid. ¹H NMR (300 MHz, CDCl₃): δ 8.42 (d, J=5.5 Hz, 1H), 6.97 (dd, J=2.0, 0.5 Hz, 1H), 6.83 (dd, J=5.5, 2.0 Hz, 1H), 4.72 (s, 2H), 3.93 (s, 1H). ¹³C NMR (75 MHz, CDCl₃): δ 161.8, 149.9, 149.7, 113.0, 110.7, 64.3. HRMS (APPI) calcd. for C₆H₇N₄O+[M+H]′ 151.0614, found: 151.0601.

### 4-Azido-2-(chloromethyl)pyridine (8)

Compound 7 (398 mg, 2.65 mmol) was solubilized in dry chloroform (3 ml) at 0° C., followed by the dropwise addition of thionyl chloride (946 mg, 7.95 mmol).

The reaction mixture was allowed to reach room temperature and was stirred for 16 h. The pH was adjusted to 8 by slow addition of saturated aq. NaHCO₃. The product was extracted with chloroform (3×3 ml). The combined organic layer was washed with brine (4 ml), dried over anhydrous MgSO₄, filtered and concentrated to dryness under reduced pressure to afford 8 (313 mg, 70%) as a yellow oil.

¹H NMR (300 MHz, CDCl₃): δ 8.48 (d, J=5.5 Hz, 1H), 7.14 (d, J=2.0 Hz, 1H), 6.89 (dd, J=5.5, 2.0 Hz, 1H), 4.64 (s, 2H). ¹³C NMR (75 MHz, CDCl₃): δ 158.7, 150.8, 150.0, 113.5, 113.1, 46.3. HRMS (APPI) calcd. for C₆H₆ClN₄⁺[M+H]⁺ 169.0276, found: 169.0284.

### 2-((4-Azidopyridin-2-yl)methyl)isoindoline-1,3-dione (9)

Potassium phthalimide (378 mg, 2.04 mmol) and 8 (313 mg, 1.85 mmol) were suspended in a solution of dimethylformamide (2 ml). The reaction mixture was stirred at room temperature for 16 h. After this time, the mixture was concentrated to dryness under reduced pressure. The solid residue was washed with water (2×2 ml) and collected by filtration to yield 9 (483 mg, 93%) as a beige solid. ¹H NMR (300 MHz, CDCl₃): δ 8.44 (d, J=5.5 Hz, 1H), 7.92-7.86 (m, 2H), 7.77-7.71 (m, 2H), 6.91 (d, J=2.0 Hz, 1H), 6.84 (dd, J=5.5, 2.0 Hz, 1H), 4.98 (s, 2H). ¹³C NMR (75 MHz, CDCl₃): δ 168.2, 157.4, 151.1, 149.6, 134.3, 132.2, 123.7, 113.0, 112.2, 42.9. HRMS (ESI-TOF) calcd. for C₁₄H₁₀N₃O₂⁺ [M+H]′ 280.0829, found: 280.0817.

### (4-azidopyridin-2-yl)methanamine (10)

To a solution of 9 (712 mg, 2.55 mmol) in tetrahydrofuran (2.7 ml) and methanol (2.7 ml) was added dropwise a solution of hydrazine hydrate (140 mg, 2.8 mmol) in methanol (0.85 ml). The reaction mixture was stirred at room temperature for 16 h. After this time, the mixture was concentrated to dryness under reduced pressure. The crude residue was purified by flash chromatography (dichloromethane/methanol, 95:5) to afford 10 (139 mg, 36%) as a yellow oil. ¹H NMR (300 MHz, CDCl₃): δ 8.46 (d, J=5.5 Hz, 1H), 6.98 (d, J=2.0 Hz, 1H), 6.82 (dd, J=5.5, 2.0 Hz, 1H), 3.97 (s, 2H), 1.77 (br s, 2H). ¹³C NMR (75 MHz, CDCl₃): δ 164.0, 150.7, 149.4, 112.4, 111.5, 47.7. HRMS (APPI) calcd. for C₆H₈N₅⁺[M+H]⁺ 150.0774, found: 150.0764.

### Azidocycloplatin or 2-aminomethylpyridine(dichloro)platinium(II)azide (11)

10 (139 mg, 0.93 mmol) was solubilized in water (9.3 ml) and the pH was adjusted to 6 by slow addition of HCl (1 N). To the resulting solution was added a solution of K₂PtCl₄ (386 mg, 0.93 mmol) in water (9.3 ml). The mixture was stirred at room temperature for 3 h. A yellow/orange precipitate formed as the reaction took place and the pH dropped to 1. The pH was adjusted to 6 by addition of NaOH (1 N). After completion of the reaction, the precipitate was collected by filtration and washed with water (2×2 ml) and ethanol (2×2 ml). The solid residue was dried in a dessicator to yield 11 (211 mg, 54%) as a yellow-orange solid. ¹H NMR (500 MHz, DMF-d₇): δ 9.10 (d, J=6.5 Hz, 1H), 7.51 (d, J=2.0 Hz, 1H), 7.30 (dd, J=6.5, 2.0 Hz, 1H), 6.23 (br s, 2H), 4.33 (t, J=6.0 Hz, 2H). ¹³C NMR (125 MHz, DMF-d₇): δ 168.7, 151.9, 149.3, 115.8, 113.1, 54.1. ¹⁹⁵Pt (107 MHz, DMF-d₇): δ 2086. HRMS (ESI-TOF) calcd. for C₇H₈Cl₂N₅O₂Pt⁻ [M+HCO₂H−H]⁻ 458.9708, found: 458.9725.

### Cycloplatin (12)

Compound 12 was prepared according to a previously published procedure (Brunner, H. & Schellerer, K.-M. Inorg. Chim. Acta 350, 39-48 (2003)). The commercially available 2-picolylamine (78 mg, 0.72 mmol) was solubilized in water (7.2 ml) and the pH was adjusted to 6 by slow addition of HCl (1 N). To the resulting solution was added a solution of K₂PtCl₄ (300 mg, 0.72 mmol) in water (7.2 ml). The mixture was stirred at room temperature for 4 h. A yellow precipitate formed as the reaction took place and the pH dropped to 1. The pH was adjusted to 6 by addition of NaOH (1 N). After completion of the reaction, the precipitate was collected by filtration and washed with water (2×2 ml) and ethanol (2×2 ml). The solid residue was dried in a dessicator to yield 12 (111 mg, 41%) as a yellow solid. ¹H NMR (300 MHz, DMF-d₇): δ 9.25 (d, J=6.5 Hz, 1H), 8.19 (td, J=7.5, 1.5 Hz, 1H), 7.73 (d, J=7.5 Hz, 1H), 7.53 (t, J=6.5 Hz, 1H), 6.25 (br s, 2H), 4.37 (t, J=6.0 Hz, 2H).

### 2-aminomethylpyridine (oxalo) platinum (II) azide APPOA (13)

11 (10 mg, 0.024 mmol) was solubilized in acetone (2 ml). Sodium oxalate (3.2 mg, 0,024 mmol) was added to the resulting solution. The mixture was stirred at 40° C. for 5 h. After completion of the reaction, the precipitate was filtrated to remove the sodium chloride salt. The filtrate was collected, concentrated to dryness under reduced pressure, and purified by HPLC (Xbridge Prep C18, 5 μm, 30×150 mm, flow rate: 30 ml/min, linear gradient: 0.1% TFA-H2O (A) and 0.1% TA-CH3CN (B), method: 0 to 50% B for 30 min, detection at 210 nm) to afford 13 (3.2 mg, 32%) as a pale yellow solid. ¹H NMR (300 MHz, DMF-d₇): δ 8,27 (d, J=5.7 Hz, 1H), 7.40 (d, J=2.2 Hz, 1H), 7.16 (dd, J=5, 7, 1.2 Hz, 1H), 6.64 (br s, 2H), 4.21 (t, J=11.2 Hz, 2H). ESI-MS calcd. for C₈H₇N₅O₄Pt⁻ [M+H]⁻ 433.01, found: 433.09.

### Cell Lines and Culture Conditions.

U2OS cells and HCT116 were cultured in standard conditions in medium supplemented with 10% fetal bovine serum (FBS), 2 mM L-glutamine, 100 U/mL penicillin and 100 μg/mL streptomycin and incubated at 37° C. with 5% CO₂. A2780 cells (cisplatin sensitive) was purchased from Sigma-Aldrich (#93112519) and maintained in RPMI-1640 medium containing 2 mM L-glutamine and 10% FBS. HCT116 RAD18 knock out cells were kindly provided by Junjie Chen's Lab (MD Anderson) and grown in DMEM medium supplemented with 10% fetal bovine serum (FBS), 2 mM L-glutamine, 100 U/mL penicillin and 100 μg/mL streptomycin.

### Cell Viability Assays.

Cell viability assays were carried out by plating U2OS cells (2,000 cells per well) in 96-well plates. Cells were treated with the relevant drug for 72 h, then incubated with CellTiter-Blue® (20 μL/well) for 1 h before recording fluorescence (560(20) Ex/590(10) Em) using a PerkinElmer Wallac 1420 Victor² Microplate Reader.

### Drugs and Inhibitors.

Picoplatin, ACP, APPOA and CP were prepared in the laboratory as described in the synthesis section of the methods. Suberoylanilide hydroxamic acid (SAHA) was purchased from Sigma and cisplatin was purchased from Tocris. Stock solutions of ACP, APPOA, picoplatin, and cisplatin were prepared at a concentration of 10 mM in DMF. A fresh stock solution of 1 mM in 0.9% w/v NaCl was freshly prepared for ACP or APPOA for use in cell imaging and pull-down experiments. Unless stated otherwise, cells were treated with ACP (250 μM), APPOA (10 μM) or cisplatin (10 μM). For co-treatments, SAHA (2.5 μM) was added to cells 2 h prior treatment with ACP, APPOA or cisplatin.

Immunofluorescence analysis and microscopy.

U2OS cells treated with ACP and/or SAHA or with APPOA and/or SAHA at ˜70% confluence. After treatments, cells were washed with PBS and pre-extracted with CSK buffer (10 mM Pipes, pH 7.0, 100 mM NaCl, 300 mM sucrose, 3 mM MgCl₂, and 0.7% Triton X-100) twice for 3 min. Then, cells were washed with PBS and fixed with 2% PFA for 13 min. In cellulo, ACP or APPOA click-labeling with Alexa Fluor® 488 alkyne (Life Technologies; #A10267) was performed based on a previously published procedure (Britton, S., et al. J. Cell Biol. 202, 575-579 (2013)). Cells were blocked and incubated for 1 h at room temperature with primary antibodies as indicated; PCNA (Abcam; ab18197), TRF1 (Abcam; ab10579), CENPA (Abcam; ab13939). The RAD18 (Abcam; ab57447) and Fibrillarin (Cell Signaling; 2639S) antibodies were incubated for 16 h at 4° C. After incubation with primary antibodies, cells were washed with PBS and incubated with the appropriate goat or rabbit secondary antibody coupled with Alexa Fluor® 647 (Life Technologies; #A-21236 or #A-21245) or rabbit secondary antibody coupled with Alexa Fluor® 594 (Life Technologies; #A-11037) in the blocking solution of each primary antibody. After PBS washes, coverslips were dipped in water and mounted on glass slides using Citifluor™ AF2 (Biovalley) or Vectashield containing DAPI (Vector laboratories) or Hoechst 33258 to visualize cell nuclei. Images were taken with Leica SP8 inverted confocal microscope, or Fluoview 1000 confocal microscope (Olympus). Data were analyzed with ImagaJ.

In Vitro Reaction of Hairpin DNA with ACP and DIBO-Alexa 488.

Hairpin (hp) DNA (5′-AAAACCAAAAATTTTTTTTTGGTTTT-3′ (SEQ ID No 1)) was diluted in 10 mM Na₂PO₄, pH 7.0, 100 mM NaNO₃, 1 mM Mg(NO₃)₂ (80 μM) and heated up at 90° C. for 5 min, then left to cool down at room temperature overnight. A stock solution of ACP at a concentration of 640 μM in 0.9% w/v NaCl was freshly prepared and reacted with an equal volume of hairpin DNA solution (typically 8 nmol). The reaction of hp with ACP was performed at 37° C. for 18 h. Unbound ACP and salts were removed using a Sephadex G-25 Medium size exclusion resin (GE Healthcare) on laboratory prepared spin columns (BioRad). Platinated DNA (hp-Pt) was reacted with DIBO-Alexa 488 (Life Technologies; #C-10405; 2.5 μl, 1.25 mM) at room temperature for 3 h. Unreacted DIBO-Alexa 488 was removed by Sephadex G-25 Medium size columns and further desalting was achieved by means of C18 ZipTips.

### MALDI-TOF Mass Spectrometry Analysis.

The ALEXA 488 labelled platinated DNA was diluted (1:9) to the matrix solution (1.7 mg of ammonium citrate to 200 μL of a saturated solution of 3-hydroxypicolinic acid (3-HPA) in acetonitrile/water (1:1 (vol/vol)). The mixture was deposited on the MALDI plate and left to dry slowly at room temperature. A MALDI-TOF/TOF UltrafleXtreme mass spectrometer (Bruker Daltonics, Bremen) was used for the experiment. Mass spectra were obtained in linear positive ion mode. All data were processed using the FlexAnalysis software package (Bruker Daltonics).

### DNA Pull-Down Assay.

U2OS cells were treated with ACP alone or in combination with SAHA. After treatment, total genomic DNA of each sample was purified using DNeasy Blood and Tissue kit (Qiagen; #69506). Pure link RNaseA (Invitrogen) was used to remove RNA during genomic DNA extraction. Click reaction was performed on the isolated DNA using Biotin-PEG4 alkyne (Sigma-Aldrich; #764213) and incubated for 1 h protected from light at room temperature. The click reaction was quenched using 4 mM EDTA. The DNA was fragmented up to ˜100-350 bp size using bioruptor (Fisher Scientific) and purified using QIAquick PCR purification kit (Qiagen; #28106). To capture the biotin tagged ACP-DNA conjugates, each sample was incubated with Dynabeads® MyOne™ Streptavidin T1 (Invitrogen, #65602) followed by washing with a buffer containing 1 M NaCl, 5 mM Tris-HCl, pH 7.5 and 0.5 mM EDTA. Beads were then washed with 8 M urea followed by three washes using the above washing buffer with 100 mM NaCl. After washing, beads were incubated in 1.8 M thiourea for 48 h at 37° C. DNA was purified using QIAquick PCR purification kit (Qiagen) and quantified using Qubit.

### RNA-Seq Sample Preparation.

Total RNA was Extracted from Cells Untreated or Treated with Acp Alone, SAHA alone or in combination of SAHA and ACP using RNeasy Mini Kit (Qiagen, #74106) following the manufacturer's protocol. Residual DNA was removed by DNase I on column digestion. RNA concentration was determined using Nanodrop and sent for RNA-seq library preparation and deep sequencing at the NGS facility, MD Anderson Cancer Center. All datasets were analyzed with FastQC to confirm a lack of sequencing abnormalities. No adapter contamination was detected. rRNA and tRNA sequences were filtered, and remaining sequences were aligned to the most recent build of the human genome (hg38) using Tophat2/Bowtie2 with sensitive parameters. Alignments with a mapping quality score of less than 5 or that were flagged as secondary were removed and files sorted and indexed. Read counts per gene were calculated from the remaining alignments using HTSeq with the Gencode v21 comprehensive genome annotation, and results were exported into a raw counts expression matrix. Differentially expressed genes were identified using edgeR with default parameters except for two modifications: first, a gene was required to have an expression value of at least 1 count per million reads in at least one sample to be tested and second, a differentially expressed gene was required to have both an absolute fold change of 1.5 or greater and a statistically significant FDR-adjusted P-value. All final results were exported to Excel and all downstream plotting was performed with custom scripts in R using the ggplot2 graphics package.

### Dot Blot Assay.

U2OS cells were treated with CP or ACP for 3 h. Total genomic DNA was isolated from cells and click reaction was performed using Alexa Fluor® 488 alkyne (Life Technologies; #A10267) followed by sonication. DNA was purified using QIAquick PCR purification kit (Qiagen, #28106) and dot blot was performed on Hybond nylon membrane (GE Healthcare). Samples were air dried and visualized using a Bio-Rad Molecular Imager ChemiDoc XRS+ system.

### Cell Lysis and Immunobloting.

Cells were washed once with PBS and lysed in lysis buffer (50 mM Tris-HCl, pH 7.5, 150 mM NaCl, 1 mM EDTA, 1% Triton X-100). For Western blot, samples were briefly sonicated followed by boiling in SDS sample buffer and separated by SDS-PAGE gels. Proteins were transferred to nitrocellulose membrane (GE Healthcare) and western blotting was performed following standard protocols. Western blots were detected by chemiluminescence (GE Healthcare Amersham ECL prime) using a Bio-Rad Molecular Imager ChemiDoc XRS+ system. The primary antibodies used for western blotting: H2AX (Millipore; #07-627), γH2AX [pSer139] (Novus Biologicals; NB100-384), histone H4 (Abcam; ab7311), acetyl-histone H4 (Lys16) (Cell Signaling; #8804), acetyl-histone H4 (Millipore; #06-866), PCNA (Santa Cruz Biotech; PC10), RAD18 (Cell Signaling; #21000), PARP (Cell Signaling; #9542), β-tubulin (Abcam; ab6046). Secondary antibodies used were: anti-rabbit IgG, HRP-linked (Cell Signaling; #7074), anti-mouse IgG, HRP-linked (Cell Signaling; #7076).

1-14. (canceled)

15. A compound of formula (I), (II) or (III) 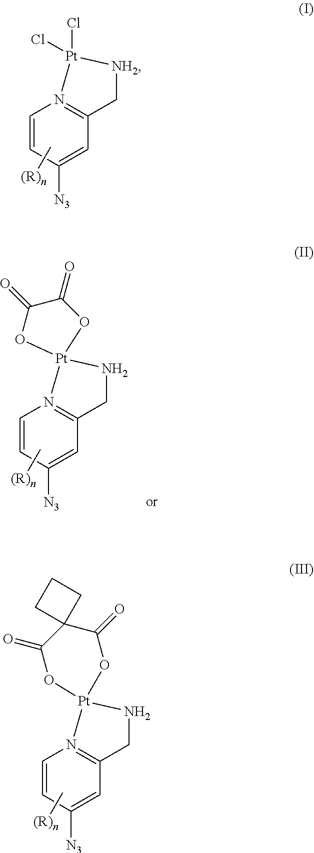
wherein n is an integer from 0 to 3 and R, independently, is selected from the group consisting of a group hydroxyl, cyano, amino, carboxyl, guanidinyl, —COOR′, —NHR′, —NR′R″, —N⁺R′R″R′″, —COR′, —CONHR′, —NHCOR′, phosphate, C(1-6) alkyl, C(2-6) alkenyl, C(1-6) alkoxy, said(1-6) alkyl, C(2-6) alkenyl, and C(1-6) alkoxy being optionally substituted by one or several groups selected from hydroxyl, cyano, amino, carboxyl, guanidinyl, —COOR′, —NHR′, —NR′R″, —N⁺R′R″R′″, —COR′, —CONHR′, —NHCOR′, aryl optionally substituted by methoxy or hydroxy, R′, R″ and R′″ being independently H or a C(1-6) alkyl.

16. The compound of claim 15, wherein n is 1 and R is in position meta in respect to N₃.

17. The compound of claim 15, wherein R is a charged radical at neutral pH.

18. The compound of claim 17, wherein the charged radical is a positively charged radical.

19. The compound of claim 15, wherein R is a C(1-6) alkyl substituted by a group selected from hydroxyl, carboxyl, amino, guanidinyl, —NHR′, —NR′R″, —N⁺R′R″R′″, —CONHR′ or an aryl, optionally substituted by a hydroxyl or a methoxy.

20. The compound of claim 15, wherein n is 0 and the formula is (I).

21. The compound of claim 15, wherein n is 0 and the formula is (II).

22. A kit comprising a compound according to claim 15 and a label bearing an alkyne group.

23. The kit of claim 22, wherein the label is a fluorescent label or a biotinylated label.

24. An in vitro method for visualizing platinated DNA crosslinks in cells, the method comprising:
contacting a cell with a compound according to claim 15;
contacting said cell with a label bearing an alkyne group, optionally in presence of copper; and
detecting the label in said cell.

25. The method of claim 24, wherein, before the step of contacting said cell with a label bearing an alkyne group, the cell is permeabilized and then fixed.

26. The method of claim 24, wherein the label is a fluorescent label.

27. An in vitro method for predicting a resistance or sensitivity of a tumor in a patient to a platinum drug, comprising:
carrying out the method according to claim 24 with a cell from a tumor sample from the patient;
measuring the labeling and optionally comparing the labeling to a reference level; and
determining the resistance or sensitivity to a platinum drug of the tumor in the patient based on the intensity of the labeling, the sensitivity being proportional to the intensity of the labeling.

28. An in vitro method for identifying or screening a molecule capable of preventing or delaying the occurrence of resistance to platinum drugs or to overcome or reduce resistance to platinum drugs, the method comprising:
contacting a cell with a compound according to claim 15 with a candidate molecule, wherein the contact with the compound can be after,
simultaneously, or before the contact with the candidate molecule;
contacting said cell with a label bearing an alkyne group, optionally in presence of copper;
measuring the labeling;
optionally comparing the intensity of the labeling in the presence and the absence of the candidate molecule;
selecting the candidate molecule if the intensity of the labeling is increased and/or the morphology of foci is different in the presence of the candidate molecule when compared to the intensity of the labeling in absence of candidate molecule.

29. The method of claim 28, wherein the label is a fluorescent label.
